(19) United States
(12) Patent Application Publication
(10) Pub. No.: US 2008/0077246 A1
(43) Pub. Date: Mar. 27, 2008

(54) VERTEBRAL DISC PROSTHESIS

(75) Inventors: Guido Fehling, Karlstein (DE); Gerald Fehling, Karlstein (DE)

Correspondence Address: AKERMAN SENTERFITT, P.O. BOX 3188, WEST PALM BEACH, FL, 33402-3188, US

(73) Assignee: FEHLING AG, Reinach (CH)

(21) Appl. No.: 11/855,738

(22) Filed: Sep. 14, 2007

(30) Foreign Application Priority Data
Sep. 21, 2006 (DE) 10 2006 045 108.2

(51) Int. Cl. A61F 2/44

(52) U.S. Cl. 623/17.16; 623/17.13; 623/17.15

(57) ABSTRACT

The implantable inter-vertebral prosthesis is comprised of a cranial endplate (10) and a caudal endplate (12), between which spring structure are provided. The spring structure are comprised of a memory metal alloy, which at body temperature exhibits super elastic characteristics. The spring structure are planar structures (14) parallel to the surface of the endplates (10, 12) and are comprised of wires of memory metal alloy tensioned running in the plane of the planar structure (14).

The invention concerns an inter-vertebral disc prosthesis.

### BACKGROUND

The inter-vertebral discs serve as elastic support upon compression between the vertebrae elements of the spinal column. Damage of the inter-vertebral discs, in particular resulting from degeneration and wear, may lead to severe limitations of mobility and neurological symptoms, in particular pain and paralysis. If such diseases can no longer be cured conservatively, it is known to replace the defective disc with an implanted inter-vertebral disc prosthesis. Such an inter-vertebral disc prosthesis is comprised of an upper cranial endplate and a lower caudal endplate, between which spring elements are introduced, which support these endplates elastically upon compression relative to each other. The inter-vertebral disc prosthesis is inserted between the vertebrae in place of the removed inter-vertebral disc, whereas the upper and lower endplates are anchored to the vertebrae elements of the superior and inferior vertebrae.

From EP 1273 276 B1 it is known to employ spring means of a memory metal alloy, preferably a nickel-titanium alloy which exhibits super-elastic properties at body temperature. The super-elasticity, which is also referred to as pseudo-elasticity, is based on a tension-induced conversion of austenite to martensite in a relatively narrow temperature range. In this temperature range, in the case of mechanical loads, the austenite was converted to a tension-inducing martensite, resulting in an elastic behavior. After the relief of tension, the austenite microstructure is again formed and the component returns to its starting condition. In the area of the super-elasticity, deformations of up to 8% with almost constant tension can be produced, so that spring means can be produced, which exhibit a constant force independent of their deformation.

In EP 1 273 276 B1, various embodiments of this spring comprised of memory metal alloy are described. In one embodiment, the spring means is formed by a nickel spring oriented axially centrally between the endplates. A helical or coil spring of this type is simple to manufacture and exhibits only small material tensions upon axial deformation. However, the achievable spring force is limited. In another embodiment, a coil spring is employed, which is provided in the shape of a toroidal ring situated between the disc ends. Thereby, greater spring forces can be achieved, the spring wire is however, subject to strong bending. In a further embodiment plate springs are employed as spring means. Thereby, high spring forces can be achieved with low construction height and the spring elements are simple to manufacture. Frictional forces, however occur between the individual spring discs, and the rotational bending occurring during spinal cord rotation cannot be spring loaded. Finally, spring means are also described, which are formed by one or more corrugated leaf springs. These springs are likewise subject to a very strong bending.

### SUMMARY

The invention is thus concerned with the task of providing a inter-vertebral disc prosthesis of the above-described generic type in which the spring means, with small axial construction height, can accept large axial loads with a low material strain. This task is inventively solved by the inter-vertebral disc prosthesis disclosed herein.

The essential concept of the invention is comprised therein, to design the spring element with at least one planar structure, which extends essentially parallel to the endplates and is comprised of memory metal alloy wires tensioned in the plain of the planar structure. This planar structure is connected with its central middle area with one of the endplates and with its radial distanced outer circumference area with the other endplate. An axial movement of the two endplates relative to each other thus leads thereto, that the central area and the circumference area of the planar structure move axially relative to each other. This axial relative movement of the central area and the circumference area results in the case of the radial separation between central area and circumference area and the conventional axial stroke movement of the endplate essentially to a longitudinal stretching of the wires and thus to a smaller bending of the wires, so that the material stretches overall and tensions remain small even on the outside of the bend. Since the spring means essentially only load the plane of the planar structure, the spring means have an extremely small axial construction height and do not influence the anatomic design of the implantable inter-vertebral prosthesis. The vertical loadability of the inter-vertebral prosthesis is translated into a horizontal stretching of the wires of the planar structure. Thereby, the relationship of the length of the wires to their stretch path optimized. With high axial reaction forces only small material tensions occur, so that the spring means are characterized by a high durability.

Basically, it is possible in accordance with the invention to employ only one planar structure as spring means, which is secured with its circumference area to the one endplate and with its central area to the other endplate. A greater axial stroke movement can be achieved in a preferred embodiment thereby, that two planar structures are employed, which respectively connected with one of the endplates and connected to a core provided between the planar structures and therewith are effectively arranged in tandem. The central area of the planar structure of the one endplate is thus connected, via the core, with the second planar structure of the other endplate. Preferably, the two planar structures are respectively connected at their circumference area with their associated endplate, while the central area of the two planar structures are connected to each other via the core located therebetween.

If the planar structures are provided with their circumference area at the endplates, then these endplates preferably exhibit a co-axial concave recess, into which the planar surface can be pressed with its central area during an axial loading. From this design there results a particularly advantageous axial construction height. The core, which connects the planar structures which during an axial loading of the planar structures is pressed into the recess of the endplates, is preferably designed co-axially convex, so that the wires of the planar structure in the case of a deflection and axial loading press against the convex bulge of the core and therewith ensure an even distribution of the bending and thus the tensional loading over the entire length of the wire. In one embodiment, the planar structure is formed of net-like crossing tensioned wires. The deflection of the wires upon axial and tensional loading is thus not uniform for all longitudinal areas of the wires and for all arrangements of the wires in the net.

In another embodiment, the planar structure is formed of radial spoke-like tensioned wires. This embodiment produces an essentially even distribution of the tension loading over the entire length of the wires in an axial deflection. At the same time, all wires are deflected in even manner in the case of a tensional loading, and cooperate in the same manner for the tensional stiffness.

In a further embodiment of the invention, the planar structures, which essentially produce the axial spring suspension of the inter-vertebral prosthesis, are surrounded on their outer circumference by elastic elements, which connect to each other the opposing outer surfaces of the endplates. These elastic elements can be formed as co-axial coil springs or as mandering spring elements distributed about the circumference. These elastic elements lead to a supplemental supporting in the case of flexing and rotation of the inter-vertebral prosthesis, that is, in the case of a tipping of the planes of the endplates relative to each other and in the case of a rotation of the endplates relative to each other.

### BRIEF DESCRIPTION OF THE DRAWINGS

In the following the invention will be described in greater detail on the basis of the illustrative embodiments shown in the figures. There is shown in:

FIG. 1 a perspective view of a inter-vertebral prostheses according to a first embodiment;

FIG. 2 an axial view upon the inter-vertebral prostheses of FIG. 1;

FIG. 3. a side view of the inter-vertebral prostheses according to a second embodiment,

FIG. 4. an axial section through the inter-vertebral prostheses of FIG. 3; and

FIG. 5. a section through the inter-vertebral prostheses of FIG. 3 according to section lines A-A in FIG. 3.

In the figures the implantable inter-vertebral prostheses is only shown schematically.

Detailed adaptation of the cross-sectional shape and the design of the endplates in the anatomic conditions of implantation are not shown, since this is not essential for the invention.

### DETAILED DESCRIPTION OF THE DRAWINGS

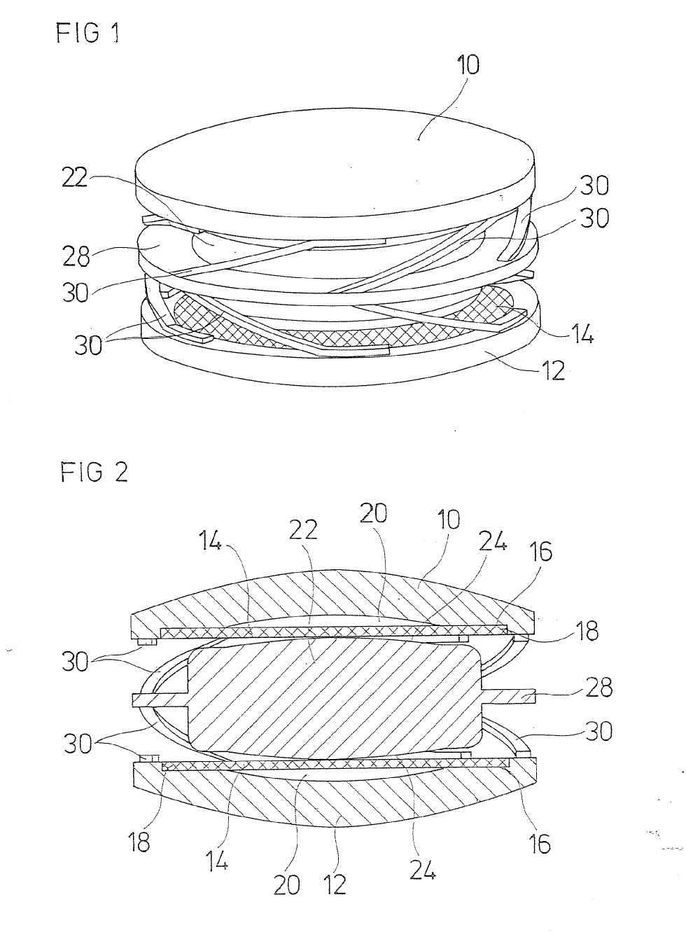

In the illustrative embodiment according to FIGS. 1 and 2 the implantable inter-vertebral prostheses and upper cranial endplate 10 and a lower caudal endplate 12. In the figures, the endplates 10 and 12 are represented as disks. An adaptation of the external contour and the respective recess of the endplates 10 and 12 to the shape of the vertebral body is of course possible. The endplates 10 and are produced of a biocompatible shape-stable material, for example, of metal or plastic.

On the surfaces of the endplates 10 and 12 facing each other, there is respectively provided a planar structure 14, which is comprised of wires of a memory metal alloy, for example, a nickel titanium alloy, which exhibits super elastic characteristics at human body temperature. In the illustrative embodiment according to FIGS. 1 and 2, the wires for forming the planar structure 14 are tensioned crossing in the manner of a net, is the case for example, in the strings of a tennis racket. The planar structure 14 of the two endplates 10 and 12 are designed identically and exhibit the shape of a circular disk. The planar structures 14 and respectively introduced into the circular shaped recess 16 of each other facing surfaces of the endplates 10 or as the case may be a 12, or in the inner circumference of the recess 16 corresponds to the outer circumference of the planar structure 14. In their outer circumference area 18, the planar structures 14 are rigidly connected with the respective endplate 10 or as the case may be a 12. The recesses 16 are designed with a concentric concave recess 10 towards their center, which is thus covered over by the planar structure 14 with axial separation increasing towards the center.

Between the planar structures 14 of the two endplates 10 and 12, there is coaxially a core 22 of a shape-stable biocompatible material, preferably of plastic. The core 22 has essentially the shape of a flat cylinder co-axial to the endplates 10 and 12. The diameter of the core 22 is somewhat smaller than the diameter of the planar structures 14. The axial end surfaces of the core 22 are provided with a concentric convex bulge 24, which is approximately complimentary in shape to the recess 20 of the endplates 10 or as the case may be 12. The core 22 is seated with its most strongly bulging center surface on the central middle area 26 of the planar structure 14 and is preferably rigidly connected in this central area 26 with the planar structure 14. In particular a form fitting junction can be produced thereby, that the core 22 is adhered or welded with the central area 26 of the planar structure 14 or that in the central area 26 a ring is introduced, which is pressed onto an axial tab or plug of the core 22.

At the outer circumference of the core 22, an outwardly projecting circular shaped flange 28 is provided in the axial center thereof. The outer circumference of the flange 28 corresponds essentially to the outer circumference of the endplate 10 and 12. Between the flange 28 and the cranial endplate 10 on the one hand and between the flange 28 and the caudal endplate 12 on the other hand elastic elements are respectively introduced, which in the illustrative embodiment according to FIGS. 1 and 2 are in the shape of coil springs 30. The coil springs 30 between the flange 28 and the cranial endplate 10 and the coil spring 30 between the flange 28 and the caudal endplate 12 are therein designed as counter rotating coil springs.

If the inter-vertebral disk prostheses implanted in the vertebral column is subject to axial load, then the endplates 10 and 12 are axially pressed together. At this time the core 22, with its bulging central area 26, presses the tensioned planar structures 14 into the recesses 20 of the endplates 10 and 12. Since the planar structures 14 are secured at their circumference area 18 to the endplates 10 or as the case may be 12, this bowing of the central area of 26 of the planar structure 14 produces a tensioning of the wires of the planar structure 14. Since the axial deflection of the center area 26 is at this time small in comparison to the radial dimensions of the planar structure 14, this bulging leads essentially to a tensioning of the wires and only to a very small bending. The bulging 24 of the outer surfaces of the core 22 brings about that the wires during increasing penetration of the core 22 into the recess 20 lies against the flat curvature of the bulge 24, so that thereby also a stronger bending of the wires is precluded. Since the wires of the planar structure 14 are therewith essentially axially stretched and only minimally bent, there results a high axial return force of the planar structure 14 with a minimal tensional loading of the material wires. This low tension loading means high durability. The flat disk-shaped design of the planar structure 14 requires only a low axial construction height due to the planar structure 14, so that the entire axial dimension of the inter-vertebral prostheses can be matched to anatomic requirements, without having to make compromises due to the spring.

Due to the ridged connection of the circumference area 18 of the planar structure 14 with endplates 10 or as the case may be 12 and the central area 26 with the core, the planar structures 14 also provide sprung support for tensional movements of the endplates 10 and 12 relative to each other.

The coil springs 30 produce on the one hand a mechanical connection between the endplates 10 and 12. Besides this, the coil springs 30 bring about a supporting of the endplates 10 and 12 relative to each other against tilting of the plains of the endplates 10 and 12 as the vertebral column bends. Finally, the coil springs 30 support tensional forces acting on the endplates 10 and 12, wherein the counter-running design of the coil springs 30 above and below the flange 28 provide a symmetrical supporting against tensional forces rotating left and rotating right.

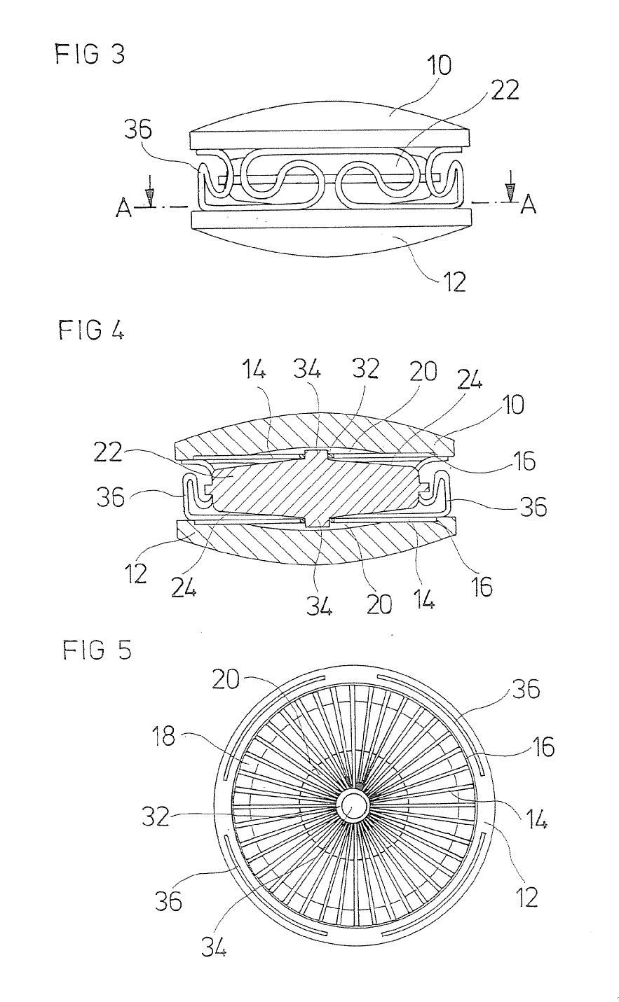

A further embodiment of the inter-vertebral prostheses is shown in FIGS. 3 through 5. To the extent that the inter-vertebral prostheses correspond with the embodiments of FIGS. 1 and 2, the same reference numbers are employed and reference can be made to the above description.

The embodiment according to FIGS. 3 through 5 differ from the illustrative embodiment according to FIGS. 1 and 2 essentially by the design of the planar structure 14. In the embodiment according to FIGS. 3 through 5, the planar structure 14 is comprised respectively of wires of a memory metal alloy, which are tensioned radially in a spoke-like manner. In the circumference area 18 the wires are secured to the respective endplate 10 or as the case may be 12, while the radial inner ends of the wires are secured in the central area 26 to a ring 32, which is secured preferably force fittingly centrally in the respective bulging surface of the core 22. In the illustrated embodiment, this ring 32 can be pressed onto a co-axial central pin or tab 34, which is provided on the surface of the core 22.

Due to the radial arrangement of the wires of the planar structure 14, in the case of a axial deflection of the planar structure 14, the wires are almost exclusively tensioned in the longitudinal direction, while the curvature due to the relationship of axial deflection to radial dimension of the planar structure 14 is minimal. Also, a torsion of a endplates 10 and 12 leads essentially to a bending of the radial wires and corresponds essentially to a tensioning of the wires in a longitudinal direction.

Further, for the supporting of the endplates 10 and 12 on their outer-circumference in the illustrative embodiment according to FIGS. 3 through 5, an alternative to the coil spring 30 and the flange 28 of the illustrative embodiments according to FIGS. 1 and 2 is shown. Between the edges of the endplates 10 and 12, projecting radially beyond the core 22 and the planar structure 14, there is employed in the illustrative example according to FIGS. 3 through 5 as elastic element a meandering bent spring wire 36 running in the circumferential direction. The sequential ends of the spring wire 36 in the circumferential direction respectively there have the shape of a laying-down “S”, which is mirror symmetric to the adjacent “S” in the circumference direction, as can be seen in FIG. 3. The meandering bent spring wire 36 produces also in this embodiment a support for the endplates 10 and 12 against a tilting of their respective planes, that is, against a flexing of the vertebral column. Besides this, the spring wire 36 supports the endplates 10 and 12 against tensional forces, wherein the sequential mirror-symmetric S shape meanderings produce a symmetric supporting against right and left rotational tensional forces.

It is also conceivable that the spring structure between the endplates 10 and 12 on the outer-circumference can be clad and enclosed by a suitable material. Such a jacketing or coating is not shown in the figures, since it is not essential for the inventive spring support of the inter-vertebral prostheses.

The total dimensioning of the inter-vertebral prostheses can be adapted or conformed to the anatomic environment of the implant situation. Representative dimensions therein would be a diameter of approximately 28 mm and a axial height of approximately 10 mm. The endplates 10 and 12 can herein have an axial edge height at the outer-circumference of approximately 1 mm. Therein there results an axial gap separation of the endplates 10 and 12 of approximately 8 mm. The axial spring path of the endplates 10 and 12 can therein be approximately maximally 1.5 mm. The axial return force of the planar structures 14 increases therein from approximately 100 N, at an axial stroke of 0.7 mm, up to approximately 2000 N, at an axial stroke of 1.5 mm.

### REFERENCE NUMBER LIST

10 Cranial Endplate
12 Caudal Endplate
14 Planar structure
16 Recess
18 Circumference Area
20 Recess
22 Core
24 Bulge
26 Central Area
28 Flange
30 Coil Spring
32 Ring
34 Tab or Pin
36 Spring Wire

1. An inter-vertebral prosthesis, comprising:
a cranial endplate (10);
an caudal endplate (12), arranged in an axial distance from the cranial endplate;
spring structure provided between the cranial endplate (10) and the caudal endplate (12), the spring structure supporting the cranial endplate (10) and the caudal endplate (12) elastically against each other, the spring structure being comprised of a memory metal alloy, which exhibits super elastic characteristics at body temperature,
wherein the spring structure comprised of at least one planar structure (14) which is substantially flat and parallel to the cranial endplate (10) and the caudal endplate (12), which is comprised of wires of memory-metal alloy tensioned in the plane of the planar structure (14),
wherein a central middle area (26) of the planar structure (14) is connected with one of said endplates (10) and an outer-circumferential area (18), that is radially distanced from the central area (26), is connected with the other of said endplates (12), and that the central middle area (26) and the outer-circumference area (18) of the planar structure (14) are capable of deflection relative to each other in an axial direction.

2. The inter-vertebral prosthesis according to claim 1, wherein one planar structure (14) is connected with the cranial endplate (10) and a second planar structure is connected with the caudal endplate (12) and that a core (22), which is shape stable and positioned substantially coaxial to said two planar structures (14), joins the two planar structures (14).

3. The inter-vertebral prosthesis according to claim 2, wherein said two planar structures (14) are connected at the circumferential area (18) with the cranial endplate (10) and the caudal endplate (12) respectively and at the central area (26) with the core (22), force fittingly.

4. The inter-vertebral prosthesis according to one of claim 1, wherein the endplate (10 or 12) that is connected to the circumferential area (18) of the planar structure (14) exhibits a concentric concave recess (20) in which the central area (26) of the planar structure (14) can be pressed in axially.

5. The inter-vertebral prosthesis according to claim 1, wherein the endplate connected at the central area (26) of the planar structure (14) or the core (22) connected at the central area (26), exhibits a concentric convex bulge (24) against which the planar structure (14) lies in the case of axial pressure load.

6. The inter-vertebral prosthesis according to claim 1, wherein the planar structure (14) is formed of net-like crossing tensioned wires.

7. The inter-vertebral prosthesis according to claim 1, wherein the planar structure (14) is formed of radial spoke-like tensioned wires.

8. The inter-vertebral prosthesis according to claim 1, wherein opposing outer edges of the cranial endplate (10) and the caudal endplate (12) are connected to each other via elastic elements, which coaxially surround the spring structure.

9. The inter-vertebral prosthesis according to claim 8, wherein the elastic element is a helical coiled spring (30) coaxial to the endplates (10, 12).

10. The inter-vertebral prosthesis according to claim 9, wherein counter thread helical rotating coil springs (30) are provided.

11. The inter-vertebral prosthesis according to claim 8, wherein the elastic element comprises a meandering bent spring wire (36) running in the circumferential direction.

12. The inter-vertebral prosthesis according to claim 2, wherein the endplate connected at the central area (26) of the planar structure (14) or the core (22) connected at the central area (26), exhibits a concentric convex bulge (24) against which the planar structure (14) lies in the case of axial pressure load.
